(19) United States
(12) Patent Application Publication
(10) Pub. No.: US 2004/0121028 A1
(43) Pub. Date: Jun. 24, 2004

(54) Plant based agents as bioavailability / bioefficacy enhancers for drugs and nutraceuticals

(75) Inventors: Ghulam Nabi Qazi, Jammu (IN); Kasturi Lal Bedi, Jammu (IN); Rakesh Kamal Johri, Jammu (IN); Subhash Chander Sharma, Jammu (IN); Manoj Kumar Tikoo, Jammu (IN); Ashok Kumar Tikoo, Jammu (IN); Sheikh Tasaduq Abdullah, Jammu (IN); Kuldip Singh, Jammu (IN); Rashmi Pandita, Jammu (IN); Om Parkash Suri, Jammu (IN); Bishan Datt Gupta, Jammu (IN); Krishan Avtar Suri, Jammu (IN); Naresh Kumar Satti, Jammu (IN); Ravi Kant Khajuria, Jammu (IN); Bal Krishan Kapahi, Jammu (IN); Ashok Kumar Kalsotra, Jammu (IN); Neelam Verma, Jammu (IN); Rajneesh Anand, Jammu (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/621,038

(22) Filed: Jul. 16, 2003

Related U.S. Application Data
(60) Provisional application No. 60/396,654, filed on Jul. 18, 2002.

(51) Int. Cl. A61K 35/78; A61K 31/7048

(52) U.S. Cl. 424/756; 514/27

(57) ABSTRACT

The invention relates to the isolation and preparation of an active fraction from plant Cuminum cyminum, its further purification and standardization as chemically defined entity (ies) with their intended use as drug bioavailability enhancer for the drugs belonging to therapeutic categories such as antimicrobial, antifungal, anti-viral, antitubercular, antileprosy, anti-inflammatory, antiarthritic, cardiovascular, antihistaminics, respiratory distress relieving drugs, immunosuppressants, anti-ulcerogenic, anti-cancer, CNS drugs, corticosteroids, nutraceuticals in compositions to be administered orally/parenterally, topically, inhalations (including nebulizers), rectally, vaginally in human beings and/or veterinary conditions.

### FIELD OF THE INVENTION

The present invention is directed to isolation/preparation of an active molecule and/or a fraction from the plant Cuminum cyminum which includes their isolation, purification and characterisation and methods of using such products to enhance bioavailability of drugs, natural products and essential nutraceuticals. The present invention is intended to enhance the bioavailability/bioefficacy of drugs which are poorly bioavailable or given for a long period of time and are expensive and toxic. The present invention also relates to the use of bioavailability enhancers—also termed as bioenhancers or BE and methods of their preparation which include their isolation from a natural source and obtaining the final product in the form of a pure isolate and/or a fraction with all the components in a chemically characterized or their fingerprint profiled form.

### BACKGROUND OF THE INVENTION

There is a great interest and medical need for the improvement of bioavailability of a large number of drugs which are (a) poorly bioavailable, (b) given for long periods, and are (c) toxic and expensive. Maximizing oral bioavailability is therapeutically important because the extent of bioavailability directly influences plasma concentrations as well as therapeutic and toxic effects resulting after oral drug administration. Poorly bioavailable drugs remain sub-therapeutic because a major portion of a dose never reaches the plasma or exerts its pharmacological effect unless and until very large doses are given which may lead to serious side effects. Any significant improvement in bioavailability will result in lowering the dose or the dose frequency of that particular drug. Besides, inter-subject variability is inversely correlated with the extent of bioavailability. Therefore, low oral bioavailability leads to high variability and poor control of plasma concentration and pharmacodynamic effects. Inter-subject variability is particularly of concern for a drug with a narrow safety margin.

Incomplete oral bioavailability has various causes. These include poor dissolution or low aqueous solubility, poor intestinal membrane permeation, degradation of the drug in gastric or intestinal fluids and pre-systemic intestinal or hepatic metabolism. The normal practice to offset some of these problems has been to increase the dosage as stated earlier which has the concerns of patients' non-compliance and toxicity.

Many therapeutic treatments are also accompanied by loss of essential nutraceuticals in the course of therapy. The present invention improves nutritional status by increasing bioavailability/bioefficacy of various nutraceuticals also which include metals and vitamins.

### DESCRIPTION OF RELATED ART

Several approaches have been adopted in the past to maximize oral bioavailability, such as (a) micronization, (b) polymorphic or crystal size and form selection, (c) solubilization of lesser soluble drugs by way of chemical modifications, complexation and use of co-solvents/surfactants, (d) targeted delivery of drug at the site of action, (e) controlled drug delivery by film coating or use of polymeric matrices for sustained release of drugs, (f) prodrug approach, and (g) microencapsulation using liposomes.

However, based on clues from Ayurvedic literature, a new approach of increasing the bioavailability of drugs including poorly bioavailable drugs had been conceptualized at RRI Jammu. One of the groups of herbals which has been documented very frequently as essential part of about 70% of Ayurvedic prescriptions, was noted to be ‘Trikatu’, that comprises the three acrids viz. long pepper, black pepper and dry ginger in equal proportions. A single major alkaloidal constituent from peppers (piperine) was found to be responsible for bioavailability enhancing effect. Influence of piperine was extensively studied on anti-TB drugs. It was determined that in combination with piperine the dose of rifampicin can be reduced by about 50% while retaining the therapeutic efficacy of this anti-TB drug at par with the standard dose (450 mg). Based on these findings several other reputed plants were evaluated for bioavailability/bioefficacy enhancing activity. Polar and non-polar extracts of parts of a few plants viz., Zingiber officinalis, Carum carvi and Cuminum cyminum increased significantly (25-300%), the bioavailability of a number of classes of drugs, for example, but not limited to, antibiotics, antifungals, anti-virals, anticancer, cardiovascular, CNS, anti-inflammatory/anti-arthritic, anti-TB/antileprosy, anti-histaminic/, corticosteroids, immunosppressants. Such extracts either in presence or absence of piperine have been found to be highly selective in their bioavailability/bioefficacy enhancing action.

### BRIEF DESCRIPTION OF THE DRAWINGS

FIG. 1 shows the chemical name and structure of the active molecule of the invention;

FIG. 2 shows an HPLC fingerprint of the active molecule; and

FIG. 3 sows an HPCL fingerprint of a fraction from the plant Cuminum cyminum.

### DESCRIPTION OF THE PREFERRED EMBODIMENT

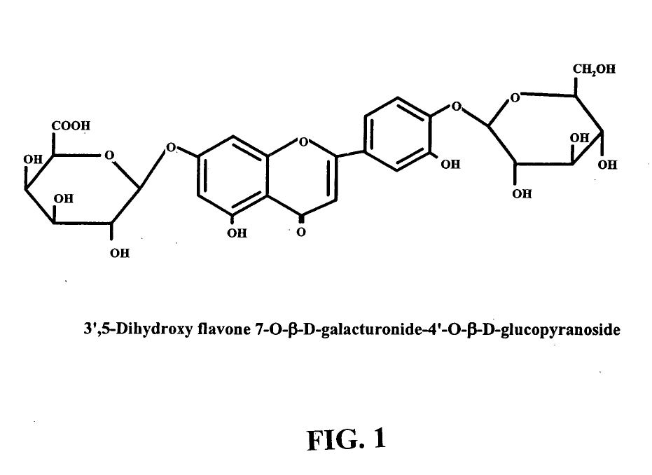

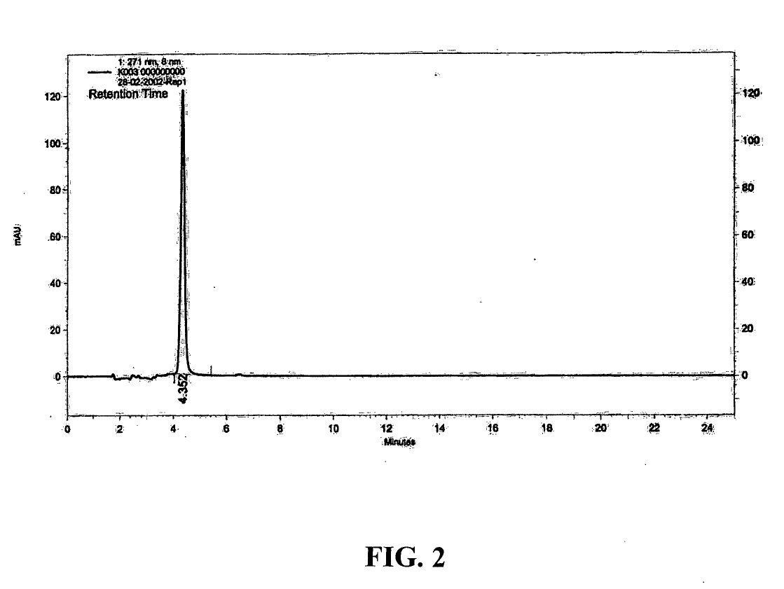

The present invention is directed to isolation/preparation of an active molecule and a fraction from the plant Cuminum cyminum which includes their isolation, purification and characterisation and methods of using such products to enhance bioavailability of drugs, natural products and essential nutraceuticals. The products of the present invention viz., an active molecule and a fraction enhances bioavailability/bioefficacy of certain drugs, natural products and essential nutraceuticals. The compound (FIG. 1) though known has been for the first time reported to be useful as an effective bioavailability enhancer. The HPLC fingerprint of FIG. 2 was done on the following sample:

| | |
|---|---|
| Sample | 3′,5-Dihydroxy flavone-7-O-β-D-galacturonide-4″-O- |
| | β-D-glucopyranoside |
| Concentration | 0.0024 gm/10 mL H₂O |
| Inj. Vol. | 5 μL |
| Column | RP-18, 5 μm |
| Mobile Phase | 2% acetic acid in H₂O:ACN (83:17) |
| Flow rate | 1 mL/min. |

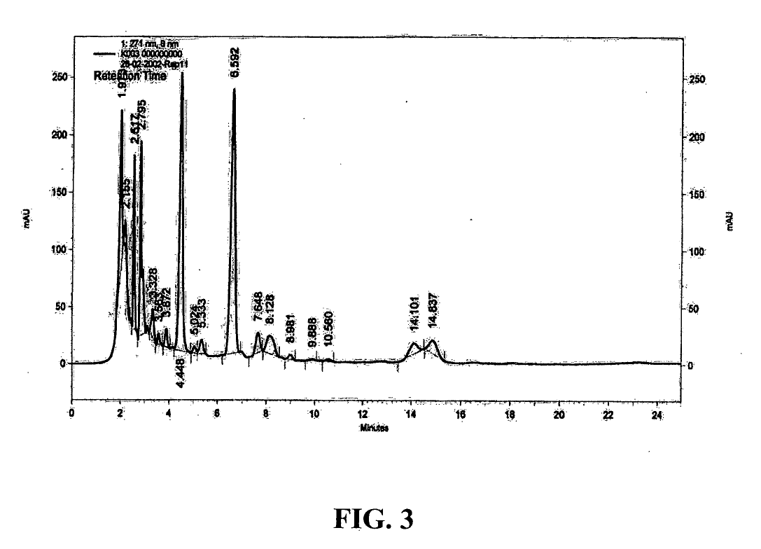

The HPLC fingerprint of FIG. 3 was done on the following sample:

| | |
|---|---|
| Sample | Fraction |
| Concentration | 0.0752 gm/10 mL H₂O |
| Inj. Vol. | 30 μL |
| Column: | RP-18, 5 μm |
| Mobile Phase | 2% acetic acid in H₂O:ACN (83:17) |
| Flow rate | 1 mL/min. |

Greenish yellow powder (H₂O:EtOH), soluble in H₂O, m.p. 270° C. decompose.

UV λmax. nm

| | |
|---|---|
| MeOH | 256.5, 267.5 sh, 350 |
| NaOMe | 265.5, 393, 5 |
| AlCl₃ | 273, 327.5 sh, 429.5 |

Physical and chemical data of as 3′,5-Dihydroxy flavone-7-O-β-D-galacturonide-4′-O-β-D-glucopyranoside

Greenish yellow powder (H₂O:EtOH), soluble in H₂O, m.p. 270° C. decompose.

UV λmax. nm

| MeOH | 256.5, 267.5 sh, 350 |
|---|---|
| NaOMe | 265.5, 393, 5 |
| AlCl₃ | 273, 327.5 sh, 429.5 |
| AlCl₃/HCl | 266, 358 |
| NaOAc | 261.5, 406.5 |
| NaOAc/H₃BO₃ | 260, 374.5 |

¹HNMR (DM SO-d6): δ 3.08-3.75 (m, 17H, sugar protons), 4.50 (d, 1H, J=7.21 Hz, H-1′″), 5.21 (d, 1H, J=6.82 Hz, H-1″), 6.42 (bs, 1H, H-6), 6.65 (bs, 1H, H-8), 6.81 (d, 1H, J=8.42, H-5′), 7.09 (s, 1H, H-3), 7.35 (q, 1H, J=8.42 and 1.8 Hz, H-6′), 7.80 (bs, 1H, H-2′).

¹³CNMR (H₂O—CD₃OD): δ 165.36 (C-2), 104.01 (C-3), 183.19 (C-4), 160.87 (C-5), 99.41 (C-6), 163.09 (C-7), 96.31 (C-8), 157.65 (C-9), 106.50 (C-10), 122.36 (C-1′), 114.10 (C-2′), 145.37 (C-3′), 148.74 (C-4′), 116.81 (C-5′), 120.75 (C-6′), 101.21 (C-1″), 73.25 (C-2″), 77.23 (C-3″), 70.72 (C-4″), 76.89 (C-5″), 62.02 (C-6″), 103.39 (C-1′″), 75.02 (C-2′″ and C-4′″), 77.71 (C-3′″) 82.07 (C-5′″), 176.44 (C-6′″).

On the basis of above data the compound has been identified as 3′,5-Dihydroxy flavone-7-O-β-D-galacturonide-4′-O-β-D-glucopyranoside (FIG. 1).

The products of the invention act by any one or more than one of the following ways: (a) Promoting the absorption of drugs from GIT, (b) Inhibiting or reducing the rate of biotransformation of drugs in the liver or intestines, (c) Modifying the immune system in a way that the overall requirement of the drug is reduced substantially, (d) Increasing the penetration or the entry into the pathogens even where they become persistors within the macrophages such as for Mycobacterium tuberculosis and such others. This eventually ensures the enhanced killing of these organisms well secured within the places otherwise inaccessible to the active drug. (e) Inhibiting the capability of pathogens or abnormal tissue to reject the drug e.g., efflux mechanisms frequently encountered with anti-malarial, anti-cancer and anti-microbial drugs, (f) Modifying the signalling process between host and pathogen ensuring increased accessibility of the drugs to the pathogens, (g) Enhancing the binding of the drug with the receptors like proteins, DNA, RNA, etc., in the pathogen, thus potentiating and prolonging its effect leading to enhanced antibiotic activity against pathogens, (h) Besides above plausible modes of action, the bioenhancer agents may also be useful for promoting the transport of nutrients and the drugs across the blood brain barrier, which could be of immense help in the control of diseases like cerebral infections, epilepsy and other CNS problems.

Primarily, but not exclusively, the invention enhances the carrier mediated entry of drugs and also the passive diffusion and the active transport pathways in the tissue which are responsible for transporting physiological substances such as nutraceuticals to their target sites. As applicable to any mechanism of action the products of this invention contribute in a synergistic and/or additive manner so that most drugs and nutraceuticals in presence of the products described in the present art are more bioavailable as a result of one or more of these mechanisms. As a preferred embodiment, the active molecule and the fraction increase the plasma levels and bioefficacy of certain categories of drugs and nutraceuticals by 80-220% over the effect that results from normal intake of therapeutic and nutraceutical products.

The ratio (w/w) of an effective bioenhancer (fraction/active molecule) in combination with a drug/nutraceutical may vary in the range of 0.1 to 300%.

The bioavailability of drugs and nutraceuticals is also relevant to animal health besides being important for humans. The invention therefore is also intended to be used in veterinary preparations.

### EXAMPLES

The following examples are intended to demonstrate some of the preferred embodiments and in no way should be construed so as to limit the scope of the invention. Any person skilled in the art can design more formulations, which may be considered as part of the present invention.

### Example 1

Cuminum cyminum seeds (0.5 kg) were ground to a coarse powder and then extracted with deionised water at 98±1° C. for 2 hrs. Extraction process was repeated four times using total of 3.1 litres water (1×1 litre+3×0.7 Litre, four extractions). All the four extracts were pooled. The pooled extract was centrifuged, followed by vacuum filtration through a celite bed. The clear filtrate was lyophilized to get greenish yellow amorphous powder (yield 88 gm, 17.6%). The dry extract was dissolved in deionised water (500 mL) and partitioned between n-BuOH (6×500 mL) and H₂O. The n-BuOH extract was concentrated on a rotavapour under reduced pressure at 65° C. (residue 11.0 gm). n-BuOH free aqueous extract was freeze dried (residue 75.0 gm) and subjected to adsorption chromatography. Aqueous extract residue was dissolved in minimum quantity of H₂O and adsorbed on SiO₂ gel, 60-120 mesh (150 gm). Solvent was completely removed to get free flowing material. A glass column of 1:5 inch dia was packed with 100 gm SiO₂ gel, 60-120 mesh in EtOAc. The adsorbed material was charged in the column over the packed SiO₂ gel. The column was eluted with EtOAc and then with EtOH by gradually increasing the %age of H₂O in EtOH. In all 420 fractions of 70 mL each were collected and pooled on the basis of TLC pattern using BuOH (B):AcOH (A):H₂O (W) (4:1:5) as developing solvent. Spots were visualized by spraying with freshly prepared Borinate-PEG solution [1% solution of 2-aminoethyl diphenylborinate in MeOH and 5% solution of polyethylene glycol 4000 in EtOH ( mixed 1:1 v/v before spraying)]. Fraction no. 81-167 (eluted in EtOH and 10% H₂O in EtOH) showed same TLC pattern. These fractions were pooled, dried and then dissolved in minimum quantity of water. Crystallisation was carried out by the addition of EtOH in small portions, supernatant was drained off and residue was washed with aq. EtOH. Residue was repeatedly crystallized from H₂O:EtOH. A yellow powder (70 mg) soluble in H₂O was thus obtained. Compound Rf 0.28 solvent system B:A:W (4:1:5) was identified as 3′,5-dihydroxy flavone 7-O-β-D-galacturonide-4′-O-β-D-glucopyranoside.

### Example 2

Cuminum cyminum seeds (0.5 kg) were ground to a coarse powder. The powder was soaked in 50% aqueous ethanol (1.0 L) for 16 hrs. The marc was extracted three times more under same conditions using 0.7 L of extraction solvent each time. The pooled extract was clarified by vacuum filtration through a celite bed. The extract thus obtained was concentrated at 60±2° C. on a rotavapour. The EtOH free extract was lyophilized to get a greenish yellow powder (88 gm, 17.60%). 80 gm of the extract was extracted by heating on a steam bath respectively with

| | | |
|---|---|---|
| 1. | CHCl₃ | (2 × 200 mL) |
| 2. | 10% EtOH in CHCl₃ | (1 × 200) |
| 3. | 20% EtOH in CHCl₃ | (1 × 200 mL) |
| 4. | 30% EtOH in CHCl₃ | (1 × 200 mL) |
| 5. | 40% EtOH in CHCl₃ | (1 × 200 mL) |
| 6. | 50% EtOH in CHCl₃ | (1 × 200 mL) |
| 7. | 60% EtOH in CHCl₃ | (1 × 200 mL) |
| 8. | 70% EtOH in CHCl₃ | (1 × 200 mL) |
| 9. | EtOH | (6 × 200 mL) |
| 10. | EtOH + 10% H₂O | (1 × 200 mL) |

The insoluble residue left after extraction with 10% water in EtOH was then extracted at room temperature with EtOH+20% H₂O (3×500 mL). The left over fraction (25 gm) was subjected to adsorption chromatography. It was adsorbed on silica gel (60-120 mesh 70 gm). Solvent was completely removed to get free flowing material. A glass column of 1.5 inch dia was packed with 70 gm SiO₂ gel 60-120 mesh in EtOH. The adsorbed extract was charged in the column. The column was eluted with EtOH by gradually increasing the %age of H₂O. In all 94 fractions of 70 mL each were collected and pooled on the basis of TLC pattern using B:A:W (4:1:5) as developing solvent. Spots were visualized by spraying the TLC plate with Borinate PEG spray reagent. Fractions 56-80 homogeneous on TLC were pooled, dried and charged on a Sephadex LH-20 column. Column was eluted with water then with EtOH to produce two fractions of 200 and 500 mL respectively. The first fraction was purified repeatedly (three times) on Sephadex LH-20 column. TLC homogeneous fractions containing target compound were pooled and residue was repeatedly crystallized from H₂O:EtOH. A yellow powder (50 mg) soluble in water was obtained. Compound Rf 0.28, Solvent system: B:A:W (4:1:5) was identified 3′,5-dihydroxyflavone 7-O-β-D-galacturonide-4′-O-β-D-glucopyranoside.

### Example 3

Cuminum cyminum seeds (100 gm) were ground to a coarse powder. Coarse powder was extracted with deionised water at 98±1° C. for 2 hrs. Extraction process was repeated four times using total water (200+3×100 mL four extractions). All the four extracts were centrifuged, followed by vacuum filtration through celite bed. The clear filtrate was lyophilized to get a greenish yellow amorphous powder (yield 17.0 gm, 17%). Aqueous extract residue was dissolved in deionised water (100 mL) and partitioned between n-BuOH (6×100 mL) and H₂O. The n-BuOH extract was concentrated on a rotavapour under reduced pressure at 65° C. (residue 2.3 gm). n-BuOH free aqueous extract was freeze dried (residue 13.9 gm). The aqueous residue was dissolved in HPLC grade H₂O (15 mg/mL) and subjected to further purification by preparative HPLC under following conditions:

| | |
|---|---|
| Colunm: | RP-18, length 10 cm × 2 cartridge with guard |
| | column |
| Column dia: | 1.5 cm |
| Sample concentration: | 15 mg/mL |
| Injection volume: | 4 mL |
| Mobile phase: | CH₃CN:H₂O (1:9) |
| Flow rate: | 10 mL/min. |
| λmax: | 271 nm |
| Run Time: | 50 minutes |

Pooled target fraction was concentrated under reduced pressure and crystallized from H₂O:EtOH to afford a yellow powder 110 mg, compound Rf 0.28, solvent system B:A:W (4:1:5) and was identified as 3′,5-dihydroxy flavone 7-O-β-D-galacturonide-4′-O-β-D-glucopyranoside.

### Example 4:

Cuminum cyminum seeds (0.5 kg) were ground to a coarse powder. Coarse powder was defatted with pet. ether 60-80 (1.0 litre) by Soxhlet extraction for 8 hrs. Pet. ether extract was discarded. The marc was dried and then extracted with EtOH (1.0 litre ) by Soxhlet extraction for 16 hrs. The EtOH extract was also discarded. The marc was then extracted with 50% aqueous EtOH at room temperature for 16 hrs each time (Total solvent used 1 litre+4×0.5 litre, five extractions). All the five extracts were pooled, concentrated on a rotavapour (residue 67 gm). This residue was dissolved in a minimum quantity of water and adsorbed on silica gel 60-120 mesh (125 gm). A glass column of 1.5 inch dia was packed with silica gel 60-120 mesh (100 gm) in EtOH. The adsorbed extract was charged in the column. Elution was carried out with solvents by gradually increasing the %age of H₂O. Each fraction of 50 mL was collected. The fractions (148-190) eluated in 10% H₂O in EtOH were pooled and subjected to further purification by preparative HPLC using following conditions.

| | |
|---|---|
| Column: | RP-18, length 10 cm × 2 cartridge with guard |
| | column |
| Column dia: | 2.5 cm |
| Sample concentration: | 15 mg/mL |
| Injection volume: | 4 mL |
| Mobile phase: | CH₃CN:H₂O (1:9) |
| Flow rate: | 10 mL/min. |
| λmax: | 271 nm |
| Run Time: | 50 minutes |

Pooled target fraction was concentrated under reduced pressure and crystallized from H₂O:EtOH to afford a yellow powder (60 mg) soluble in water, compound Rf 0.28, solvent system B:A:W (4:1:5) and was identified as 3′,5-dihydroxyflavone 7-O-β-D-galacturonide-4′-O-β-D-glucopyranoside.

### Example 5

List of drugs cited as some of the examples for the purpose of the present invention.

| | Categories | Drugs |
|---|---|---|
| I | Antibiotics | Fluoroquinolones: |
| | | Cipro-, Nor-, P-, and 0-floxacins |
| | | Macrolides: Erythro-, Roxythro-, and |
| | | Azithromycin |
| | | Cephalosporins: Cefixime, Cefalexin, |
| | | Cefadroxil, Cefatrioxone |
| | | Penicillins: moxycillin, Cloxacillin |
| | | Aminoglycosides: Amikacin, Kanamycin |
| II. | Antifungal | Fluconazole, Amphotericin B, |
| | | Ketoconazole |
| III. | Anti-viral | Acyclovir, Zidovudine |
| IV. | Anti-cancer | Methotrexate, 5-Fluorouracil, Doxorubicin |
| | | Cisplatin |
| V. | Cardiovascular | Amlodipin, Lisinopril, Atenolol |
| VI. | CNS | Alprazolam, Haloperidol |
| VI. | Anti-inflammatory/ | Diclofenac Piroxicam, Nimesulide, |
| | antiarthritic (NSAID) | Rofecoxib |
| VII. | Anti-TB/Antileprosy | Rifampicin Ethionamide, Isoniazid, |
| | | Cycloserine, Dapsone, Pyrazinamide, |
| | | Ethambutol |
| VIII. | Anti histamines/ | Salbutamol, Theophylline, Bromhexine, |
| | respiratory disorders | Loratidine |
| IX. | Corticosteroids | Prednisolone, Dexamethasone, |
| | | Betamethasone |
| X. | Immunosuppressants | Cyclosporin A, Tacrolimus |
| | | Mycophenolate mofetil |
| XI. | Antiulcer | Ranitidine, Cimetidine, Omeprazole |

### Example 5

(i): Antibiotics:

(a) Fluroquinolones

% Enhancement in bioavailability

| Drug | Active molecule | Fraction |
|---|---|---|
| Ciprofloxacin | 65 | 130 |
| P-floxacin | 55 | 137 |
| O-floxacin | 70 | 103 |
| Norfloxacin | 45 | 55 |

(b) Macrolides

% Enhancement in bioavailability

| Drug | Active molecule | Fraction |
|---|---|---|
| Erythromycin | 70 | 80 |
| Roxythromycin | 65 | 105 |
| Azithromycin | 82 | 115 |

(c) Cephalosporins

% Enhancement in bioavailability

| Drug | Active molecule | Fraction |
|---|---|---|
| Cefalexin | 70 | 105 |
| Cefadroxil | 85 | 120 |
| Cefatrioxone | 75 | 100 |
| Cefixime | nil | Nil |

(d) Penicillins

% Enhancement in bioavailability

| Drug | Active molecule | Fraction |
|---|---|---|
| Amoxycillin | 68 | 105 |
| Cloxacillin | 77 | 105 |

(e) Aminoglycosides:

% Enhancement in bioavailability

| :Drug | Active molecule | Fraction |
|---|---|---|
| Amikacin | 76 | 87 |
| Kanamycin | Nil | 35 |

5 (ii) Antifungal

% Enhancement in bioavailability

| Drug | Active molecule | Fraction |
|---|---|---|
| Fluconazole | 110 | 105 |
| Amphotericin B | 95 | 90 |
| Ketoconazole | 77 | 85 |

5 (iii) Anti-viral

% Enhancement in bioavailability

| Drug | Active molecule | Fraction |
|---|---|---|
| Acyclovir | 89 | 110 |
| Zidovudine | 120 | 135 |

5. (iv) CNS drugs:

% Enhancement in bioavailability

| Drug | Active molecule | Fraction |
|---|---|---|
| Alprazolam | 70 | 75 |
| Haloperidol | 72 | 60 |

5. (v) Anti-cancer

% Enhancement in bioavailability

| Drug | Active molecule | Fraction |
|---|---|---|
| Methotrexate | 95 | 140 |
| 5-Fluorouracil | 110 | 240 |
| Doxorubicin | 78 | 90 |
| Cisplatin | 65 | 95 |

5. (vi) Cardiovascular:

% Enhancement in bioavailability

| Drug | Active molecule | Fraction |
|---|---|---|
| Amlodipine | 80 | 130 |
| Lisinopril | Nil | Nil |
| Atenolol | 75 | 110 |
| Propranolol | 85 | 140 |

5. (vii) Anti-inflammatory/antiarthritic:

% Enhancement in bioavailability

| Drug | Active molecule | Fraction |
|---|---|---|
| Diclofenac | 105 | 125 |
| Piroxicam | 76 | 100 |
| Nimesulide | 90 | 115 |
| Rofecoxib | 43 | 70 |

5. (viii) Anti-TB/Antileprosy drugs:

% Enhancement in bioavailability

| Drug | Active molecule | Fraction |
|---|---|---|
| Rifampicin | 90 | 170 |
| Isoniazid | Nil | 30 |
| Pyrazinamide | Nil | Nil |
| Ethambutol | Nil | Nil |
| Dapsone | 67 | 93 |
| Ethionamide | 120 | 110 |
| Cycloserine | 85 | 110 |

5. (ix) Anti-histamines/respiratory disorders:

% Enhancement in bioavailability

| Drug | Active molecule | Fraction |
|---|---|---|
| Salbutamol | 98 | 75 |
| Theophylline | 75 | 95 |
| Bromhexine | Nil | 35 |
| Loratidine | 62 | 45 |

5. (x) Corticosteroids:

% Enhancement in bioavailability

| Drug | Active molecule | Fraction |
|---|---|---|
| Prednisolone | 46 | 57 |
| Dexamethasone | 67 | 60 |
| Betamethasone | 65 | 50 |

5. (xi) Immunosuppressants:

% Enhancement in bioavailability

| Drug | Active molecule | Fraction |
|---|---|---|
| Cyclosporin A | 135 | 170 |
| Tacrolimus | 90 | 110 |
| Mycophenolate | Nil | Nil |
| Mofeit | | |

5. (xii) Anti-ulcer

% Enhancement in bioavailability.

| Drug | Active molecule | Fraction |
|---|---|---|
| Ranitidine | 95 | 95 |
| Cimetidine | 76 | 70 |
| Omeprazole | 72 | 87 |

### Example 6

Herbal formulations:

EXAMPLE 6
Herbal formulas:

% Enhancement in
bioavailability/bioefficacy

| Drug | Active molecule | Fraction |
|---|---|---|
| Echinacea | 77 | 147 |
| Tinospora cordifolia | 102 | 185 |
| Picrorrhiza kurroa | 68 | 180 |
| Aegles marmelos | Nil | Nil |
| Andrographis paniculata | Nil | 190 |
| Emblica ribes | 67 | 90 |
| Asparagus racemosus | 78 | 145 |
| Terminalia chebula | 45 | 85 |
| Withania somnifera | Nil | Nil |
| Centella asiatica | 82 | 60 |

C. Nutraceuticals

| | Category |
|---|---|
| I. | Vitamins |
| | Vitamin A |
| | Vitamin E |
| | Vit. B1 |
| | Vit. B6 |
| | Vit B12 |
| | Vit. C |
| | Folic acid |
| II | Antioxidants |
| | β-Carotene |
| | Silymarin |
| | Selenium |
| | Lycopene |
| | Ellagiogallotannins |
| III | Natural herbal products |
| | Curcumin |
| | Boswellic acids |
| | Rutin |
| IV | Essential nutritional components |
| | Methionine |
| | Lysine |
| | Leucine |
| | Valine |
| | Isoleucine |
| | Zinc |
| | Calcium |
| | Glucose |
| | Potassium |
| | Copper |
| | Iron |

1. A composition for enhancing bioavailability of drugs/nutraceuticals, said composition comprising an active drug/nutraceutical and an effective amount of bioenhancer selected from a fraction obtained from Cuminum cyminum having characteristics as shown in FIG. 3 and an active molecule of formula 1 to enhance pharmaceutical effect of said active drug/nutraceutical without any harmful side effect.

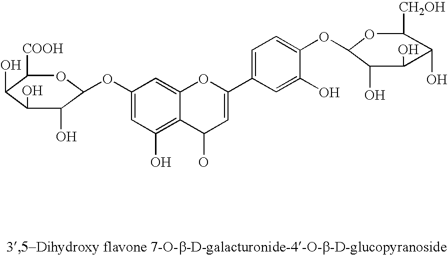

2. A composition as claimed in claim 1, wherein w/w ratio of the bioenhancer to the drug/nutraceutical is in the range of 0.1 to 300.

3. A composition as claimed in claim 1, wherein said bioenhancer increases bioavailability of the drug/nutraceutical by 80-220%.

4. A composition as claimed in claim 1, wherein said active drug is selected from the group comprising of antibiotics, anti-fungal drugs, antiviral drugs, anticancer drugs, cardiovascular disorder drugs, CNS disorders drugs, antiinflammatory/antiarthritic drugs, anti-TB/anti-leprosy drugs, anti-histamines/respiratory disorder drugs, corticosteroids, immuno-suppressants and anti ulcer drugs.

5. A composition as claimed in claim 4, wherein said antibiotic is selected from the group comprising of fluroquinolones, macrolides, cephalosporins, penicillins and aminoglycosides.

6. A composition as claimed in claim 5, wherein said fluroquinolone is selected from the group comprising of ciprofloxacin, p-floxacin, o-floxacin and norfloxacin.

7. A composition as claimed in claim 5, wherein said macrolide is selected from the group comprising of erythromycin, roxythromycin and azithromycin.

8. A composition as claimed in claim 5, wherein said cephalosporin is selected from the group comprising of cefalexin, cefadroxil and cefatrioxone.

9. A composition as claimed in claim 5, wherein said penicillin is selected from the group comprising of amoxycillin and cloxacillin.

10. A composition as claimed in claim 5, wherein said aminoglycoside used is amikacin.

11. A composition as claimed in claim 4, wherein said antifungal drug is selected from the group comprising of fluconazole, amphotericin B and ketoconazole.

12. A composition as claimed in claim 4, wherein said antiviral drug is selected from the group comprising of acyclovir and zidovudine.

13. A composition as claimed in claim 4, wherein said CNS drug is selected from the group comprising of alprazolam and haloperidol.

14. A composition as claimed in claim 4, wherein said anti-cancer drug is selected from the group comprising of methotrexate, 5-fluorouracil, doxorubicin and cisplatin.

15. A composition as claimed in claim 4, wherein said cardiovascular disorder drug is selected from the group comprising of amlodipine, atenolol and propranolol.

16. A composition as claimed in claim 4, wherein said anti-inflammatory/antiarthritic drug is selected from the group comprising of diclofenac, piroxicam, nimesulide and rofecoxib.

17. A composition as claimed in claim 4, wherein said anti-TB/antileprosy drug is selected from the group comprising of rifampicin, dapsone, ethionamide and cycloserine.

18. A composition as claimed in claim 4, wherein said anti-histamines/respiratory disorder drug is selected from the group comprising of salbutamol, theophylline and loratidine.

19. A composition as claimed in claim 4, wherein said corticosteroid is selected from the group comprising of prednisolone, dexamethasone and betamethasone.

20. A composition as claimed in claim 4, wherein said immunosuppressant is selected from the group comprising of cyclosporin A and tacrolimus.

21. A composition as claimed in claim 4, wherein said anti-ulcer drug is selected from the group comprising of ranitidine, cimetidine and omeprazole.

22. A composition as claimed in claim 1, wherein nutraceutical is selected from the group comprising of vitamins, antioxidants, natural herbal products, herbal formulations and essential nutritional components.

23. A composition as claimed in claim 22, wherein said vitamin is selected from the group comprising of Vitamin A, E, B1, B6, B12, C and Folic acid.

24. A composition as claimed in claim 22, wherein said antioxidant is selected from the group comprising of β-carotene, silymarin, selenium, lycopene and ellagiogallotannins.

25. A composition as claimed in claim 22, wherein said natural herbal product is selected from the group comprising of curcumin, boswellic acids and rutin.

26. A composition as claimed in claim 22, wherein herbal formulation is selected from the group comprising of echinacea, tinospora cordifolia, picrorrhiza kurroa, emblica ribes, asparagus racemosus, terminalia chebula and centella asiatica.

27. A composition as claimed in claim 22, wherein said nutritional component is selected from the group comprising of methionine, lysine, leucine, valine, isoleucine, zinc, calcium, glucose, potassium, copper and iron.

28. A composition as claimed in claim 1, wherein said composition is administered orally or intramuscularly and is also relevant to animal health.

29. A method for enhancing bioavailability of drugs/nutraceuticals said method comprising of admixing to the drug/nutraceutical an effective amount of bioenhancer selected from a fraction obtained from Cuminum cyminum having characteristics as shown in FIG. 3 and an active molecule of formula 1 to enhance pharmaceutical effect of said active drug/nutraceutical without any harmful side effect.

30. A method as claimed in claim 29, wherein the bioavailability of the antibiotic is enhanced by 45 to 85% when the same is mixed with the active molecule of formula 1.

31. A method as claimed in claim 29, wherein the bioavailability of the antibiotic is enhanced by 55 to 137% when the same is mixed with the fraction.

32. A method as claimed in claim 29, wherein the bioavailability of the antifungal drug is enhanced by 77 to 110% when the same is mixed with the active molecule of formula 1.

33. A method as claimed in claim 29, wherein the bioavailability of the antifungal drug is enhanced by 85 to 105% when the same is mixed with the fraction.

34. A method as claimed in claim 29, wherein the bioavailability of the antiviral drug is enhanced by 89 to 120% when the same is mixed with the active molecule of formula 1.

35. A method as claimed in claim 29, wherein the bioavailability of the antiviral drug is enhanced by 120 to 135% when the same is mixed with the fraction.

36. A method as claimed in claim 29, wherein the bioavailability of the CNS drug is enhanced by 70 to 72% when the same is mixed with the active molecule of formula 1.

37. A method as claimed in claim 29, wherein the bioavailability of the CNS drug is enhanced by 60 to 75% when the same is mixed with the fraction.

38. A method as claimed in claim 29, wherein the bioavailability of the anti-cancer drug is enhanced by 65 to 110% when the same is mixed with the active molecule of formula 1.

39. A method as claimed in claim 29, wherein the bioavailability of the anti-cancer drug is enhanced by 90 to 240% when the same is mixed with the fraction.

40. A method as claimed in claim 29, wherein the bioavailability of the cardiovascular disorder drug is enhanced by 75 to 85% when the same is mixed with the active molecule of formula 1.

41. A method as claimed in claim 29, wherein the bioavailability of the cardiovascular disorder drug is enhanced by 110 to 140% when the same is mixed with the fraction.

42. A method as claimed in claim 29, wherein the bioavailability of the anti-inflammatory/antiarthritic drug is enhanced by of 43 to 105% when the same is mixed with the active molecule of formula 1.

43. A method as claimed in claim 29, wherein the bioavailability of the anti-inflammatory/antiarthritic drug is enhanced by 70 to 125% when the same is mixed with the fraction.

44. A method as claimed in claim 29, wherein the bioavailability of the anti-TB/antileprosy drug is enhanced by of 67 to 120% when the same is mixed with the active molecule of formula 1.

45. A method as claimed in claim 29, wherein the bioavailability of anti-TB/antileprosy drug is enhanced by 93 to 170% when the same is mixed with the fraction.

46. A method as claimed in claim 29, wherein the bioavailability of the anti-histamines/respiratory disorder drug is enhanced by of 62 to 98% when the same is mixed with the active molecule of formula 1.

47. A method as claimed in claim 29, wherein the bioavailability of anti-histamines/respiratory disorder drug is enhanced by 35 to 95% when the same is mixed with the fraction.

48. A method as claimed in claim 29, wherein the bioavailability of corticosteroids is enhanced by of 46 to 67% when the same is mixed with the active molecule of formula 1.

49. A method as claimed in claim 29, wherein the bioavailability of corticosteroids is enhanced by 50 to 60% when the same is mixed with the fraction.

50. A method as claimed in claim 29, wherein the bioavailability of immunosuppressants is enhanced by of 90 to 135% when the same is mixed with the active molecule of formula 1.

51. A method as claimed in claim 29, wherein the bioavailability of immunosuppressants is enhanced by 110 to 170% when the same is mixed with the fraction.

52. A method as claimed in claim 29, wherein the bioavailability of anti-ulcer drugs is enhanced by 72 to 85% when the same is mixed with the active molecule of formula 1.

53. A method as claimed in claim 29, wherein the bioavailability of anti-ulcer drugs is enhanced by 70 to 95% when the same is mixed with the fraction.

54. A method as claimed in claim 29, wherein the bioavailability of herbal formulation is enhanced by 45 to 102% when the same is mixed with the active molecule of formula

55. A method as claimed in claim 29, wherein the bioavailability of herbal formulation is enhanced by 35 to 147% when the same is mixed with the fraction.

56. A method as claimed in claim 29, wherein said method is administered orally or intramuscularly and is also relevant to animal health.
